(19) United States
(12) Patent Application Publication
(10) Pub. No.: US 2005/0191339 A1
(43) Pub. Date: Sep. 1, 2005

(54) Skin friendly active ingredient plaster based on SBC, containing at least 34 WT.% of a pharmaceutical active ingredient and production thereof

(75) Inventors: Andreas Kummer, Hamburg (DE); Matthias Wasner, Hamburg (DE); Jens-Uwe Wuestling, Hamburg (DE)

Correspondence Address: GREENBLUM & BERNSTEIN, P.L.C., 1950 ROLAND CLARKE PLACE, RESTON, VA, 20191, US

(73) Assignee: Beiersdorf AG, Hamburg (DE)

(21) Appl. No.: 11/050,670

(22) Filed: Feb. 7, 2005

Related U.S. Application Data
(63) Continuation of application No. PCT/EP03/08709, filed on Aug. 6, 2003.

(30) Foreign Application Priority Data
Aug. 8, 2002 (DE) 102 36 319.6

(51) Int. Cl. A61K 9/70; A61F 13/00

(52) U.S. Cl. 424/449

(57) ABSTRACT

An active ingredient containing bandage for the controlled delivery of a pharmaceutically active ingredient to the skin. The bandage comprises a flexible cover layer and a water-insoluble, adhesive styrene block copolymer (SBC)-based matrix that comprises at least 34% by weight of the pharmaceutically active ingredient.

### CROSS-REFERENCE TO RELATED APPLICATIONS

The present application is a continuation of International Application No. PCT/EP2003/008709, filed Aug. 6, 2003, the entire disclosure whereof is expressly incorporated by reference herein, which claims priority under 35 U.S.C. § 119 of German Patent Application No. 102 36 319.6, filed Aug. 8, 2002.

### BACKGROUND OF THE INVENTION

1. Field of the Invention

The invention relates to an active ingredient plaster system for controlled delivery to the skin of at least one pharmaceutically active ingredient, containing at least 34 wt. %, over a prolonged period of time and also to a process for producing it.

2. Discussion of Background Information

Transdermal therapeutic systems (TTS) for delivering active ingredients through the skin have been known for a long time. The topical application of drugs by way of active ingredient bandage (or patch) systems offers two main advantages: first this form of administration produces first-order release kinetics of the active ingredient, thereby enabling a constant level of active ingredient to be maintained in the body over a very long period of time. Secondly the path of uptake through the skin avoids the gastrointestinal tract and also the first pass through the liver. As a result selected drugs can be administered effectively in a low dose. This is particularly advantageous when it is desired that the drug should act locally, avoiding a systemic effect. This is the case, for example, with the treatment of hyperkeratoses, corns, calluses and warts.

One embodiment of such transdermal systems which has been well described in the technical literature is that of matrix systems or monolithic systems in which the drug is incorporated directly into the adhesive. In the ready-to-apply product an adhesive matrix of this kind, comprising active ingredient, is provided on one side with a backing, which is occlusive for the active ingredient, while on the opposite side there is a backing film equipped with a release layer, which is removed prior to application to the skin (kleben & dichten, No. 42, 1998, pp. 26 to 30).

A fundamental requirement of a TTS is, on the one hand, effective adhesion to skin, which must be maintained over the entire duration of the intended dosing with active ingredient, and, on the other hand, the residueless removability of the TTS. Additionally, painful redetachment of the active ingredient patch following a prolonged period of wear is frequently observed. Besides adhesives which are coated in solution onto the backing, use is also made, among others, of solvent-free systems, such as hot melt adhesives. A distinguishing feature of these is that in the coating operation it is possible to forego the use of organic solvent and dispersant. As a result of heating, hot melt adhesives are converted into the liquid form and applied thus, as a melt, to the respective bandage backing. As well as technical aspects, such as solvent processing, anti-explosion design of plant, and environmental protection impositions, medical grounds, too, play a part in the choice of solvent-free adhesives. Transdermal therapeutic systems are applied generally to healthy, intact skin. Here in particular it is especially important not to irritate, let alone damage, the skin as a result of a drug. One frequently observed side effect is the appearance of skin irritations, which occur in particular when a TTS is applied for a relatively long period, or repeatedly, to the same region of the body. The principal cause of these irritations are the ingredients of the adhesive matrix. In the case of solvent borne systems it is possible, following the extraction of the solvents, to recover a residual content, which in view of the allergenic potential may likewise lead to unwanted instances of skin irritation. For application to skin above all, therefore, preference is given to solvent-free systems, whose formulations, in particular, comprise skin-friendly ingredients.

Descriptions are given of hot melt adhesives in EP-A 663 431, EP-A 542 034, EP-A 305 757, DE-A 42 22 334 and DE-C 42 24 325. Active ingredients listed in these specifications, if identified at all, are those which act systemically. A feature of systemically active TTS is that they pass the active ingredient through the skin into the bloodstream. The blood carries the active ingredient via the whole body to the site of action. Locally active TTS acts only at the site of application, since the skin is impermeable for the active ingredient. Locally active systems find application, inter alia, in thermal therapies, for skin care and for foot care.

Known for the preparation of hot melt adhesives are, among other systems, SBC polymers as a skeleton substance. One such system is described in DE 196 50 471 A1 for the application of hyperemic ingredients. Hyperemic ingredients, such as capsaicin, nonylvanillylamide or belladonna, are distinguished by the fact that even small amounts are sufficient for the target thermal therapy. Use in concentrations above 20% by weight is unknown for these active ingredient systems.

It is desirable to develop a skin-friendly active ingredient bandage system for the controlled delivery to the skin of at least one active pharmaceutical ingredient in concentrations of at least 34% by weight that meets the specified requirements of a TTS.

### SUMMARY OF THE INVENTION

The present invention provides an active ingredient containing bandage for the controlled delivery of at least one pharmaceutically active ingredient to the skin. The bandage comprises a flexible cover layer and a water-insoluble, adhesive styrene block copolymer (SBC)-based matrix that comprises at least 34% by weight of at least one pharmaceutically active ingredient, based on the total weight of the matrix composition.

In one aspect of the bandage of the present invention, the SBC may comprise an A-B block copolymer and/or an A-B-A block copolymer where A is a hard phase which comprises predominantly polystyrene and derivatives thereof and B is a soft phase which comprises predominantly units of one or more of ethylene, propylene, butylene, butadiene and isoprene. For example, the A-B block copolymer and/or A-B-A block copolymer may comprise less than 35% by weight of styrene units, e.g., from 5% to 30% by weight of styrene units, and/or less than 80% by weight of the A-B block copolymer and A-B-A block copolymer may consist of an A-B block copolymer.

In another aspect of the bandage, the matrix may comprise from 4% to 55% by weight of SBC, e.g., from 5% to 50% by weight of SBC, or from 7% to 45% by weight of SBC.

In yet another aspect, the matrix may comprise up to 70% by weight of the at least one pharmaceutically active ingredient, e.g., from 36% to 65% by weight, or from 37% to 62% by weight.

In a still further aspect, the bandage may comprise from 4% to 55% by weight of SBC, from 5% to 55% by weight of tackifiers, less than 51% by weight of plasticizers, less than 5% by weight of stabilizers, and up to 70% by weight of the at least one pharmaceutically active ingredient.

In another aspect of the bandage, the at least one pharmaceutically active ingredient may comprise an active ingredient that is locally active. For example, the active ingredient may comprise salicylic acid.

In another aspect, all components of the matrix may have been melt-homogenized without the use of solvents.

The present invention also provides a process for the solvent-free and mastication-free production of a self-adhesive styrene block copolymer (SBC)-based composition which comprises at least one pharmaceutically active ingredient in an amount of at least 34% by weight based on a total weight of SBC-based composition in a continuously operating apparatus. The process comprises
(a) feeding an initial batch which comprises SBC, at least a part of the at least one pharmaceutically active ingredient and, optionally, at least a part of any further components of the composition into the feeding section of the apparatus;
(b) transferring the initial batch from the feeding section to the compounding section of the apparatus;
(c) optionally, adding any remaining part of the at least one pharmaceutically active ingredient and any remaining part of the further components of the composition which have not been added to the feeding section to the compounding section; and
(d) producing a homogeneous self-adhesive composition in the compounding section.

In one aspect of this process, the further components of the composition may be selected from one or more of low molecular weight SBCs, fillers, plasiticizers, tackifiers, resins, release aids and additives.

In another aspect of the process, the apparatus may comprise a twin-screw extruder. For example, the twin-screw extruder may comprise at least one metering port, e.g., from two to seven metering ports, and at least one devolatilization port.

In yet another aspect of the process, the temperature inside the apparatus may be from 85° C. to 120° C., for example, up to 110° C., or up to 100° C.

The present invention also provides a process for the solvent-free and mastication-free production of a self-adhesive styrene block copolymer (SBC)-based composition which comprises at least one pharmaceutically active ingredient in an amount of at least 34% by weight based on a total weight of SBC-based composition in a continuously operating apparatus. The process comprises
(a) feeding an initial batch which comprises SBC and, optionally, at least a part of any further components of the composition with the exception of the at least one pharmaceutically active ingredient into the feeding section of the apparatus;
(b) transferring the initial batch from the feeding section to the compounding section of the apparatus;
(c) adding the at least one pharmaceutically active ingredient and, optionally, any remaining part of the further components of the composition which has not been added to the feeding section to the compounding section;
(d) producing a homogeneous self-adhesive composition in the compounding section.

In one aspect of this process, the further components of the composition may be selected from one or more of low molecular weight SBCs, fillers, plasiticizers, tackifiers, resins, release aids and additives.

In another aspect of the process, the apparatus may comprise a twin-screw extruder. For example, the twin-screw extruder may comprise at least one metering port, e.g., from two to seven metering ports, and at least one devolatilization port.

In yet another aspect of the process, the temperature inside the apparatus may be from 85° C. to 120° C., for example, up to 110° C., or up to 100° C.

The present invention also provides a process for producing a self-adhesive, active ingredient containing matrix bandage. This process comprises carrying out any of the processes set forth above, transferring the self-adhesive composition to a coating device and applying the self-adhesive composition to a carrier material selected from a web material, a release film and a release paper.

In one aspect of this process, the coating device may comprise an extrusion die and/or the coating device may comprise a roll unit or a calender unit.

In another aspect of the process, the thickness of the self-adhesive composition on the carrier material may be adjusted to from 10 μm to 2,000 μm, e.g., from 100 μm to 500 μm.

The present invention also provides an active ingredient containing self-adhesive SBC-based composition and a self-adhesive, active ingredient containing matrix bandage which are obtainable by any of the processes set forth above.

It has become apparent that SBC polymers meet the above-stated requirements. The hot melt adhesives based on SBC polymers, by virtue of their synthetic preparation, are free from unwanted ingredients and, depending on molecular weight, feature adhesion and cohesion properties which can be adjusted with the aid of tackifiers such as resins, oils and plasticizers. As a result of their thermoplastic behavior they can be processed in the melt without mastication. The thermoplastic properties of the SBC polymers also aid in the doping of active ingredient with a high recovery rate, since active ingredients can be homogeneously distributed in the adhesive melt, even at high concentrations, at relatively low temperatures and with relatively low mixing times, and by means of rapid cooling it is possible to reduce the degradation of thermally sensitive active ingredients as compared with solvent systems, since there are no long drying times for the evaporation of the dispersants and/or solvents.

The SBC polymers are preferably A-B or A-B-A block copolymers or mixtures thereof, the hard phase, A, comprising predominantly polystyrene or derivatives thereof and the soft phase, B, comprising ethylene, propylene, butylene, butadiene, isoprene or mixtures thereof. Polystyrene blocks, however, may also be present in the soft phase in concentrations of up to 20%.

The invention accordingly provides with preference an active ingredient matrix bandage for the controlled delivery to the skin of active ingredients, which is composed of a flexible top layer and a water-insoluble, adhesive matrix comprising active ingredient, the adhesive matrix being composed of:

| | |
|---|---|
| A | block copolymers at 4 to 55 wt. %, |
| B | tackifiers at 5 to 55 wt. %, |
| C | plasticizers at less than 51 wt. %, |
| D | stabilizers at less than 5 wt. %, |
| E | active ingredients at 34 to 70 wt. % |

A typical adhesive of the invention is therefore composed of the following components:

| | | | | |
|---|---|---|---|---|
| A | 5-50 | wt. % | preferably 7-45 | wt. % |
| B | 7-52 | wt. % | preferably 10-50 | wt. % |
| C | 2-50 | wt. % | preferably 4-40 | wt. % |
| D | 0-5 | wt. % | preferably 0.2-3 | wt. % |
| E | 34-70 | wt. % | preferably 36-65 | wt. % |
| | | | more preferably 37-62 | wt. % |

Optionally it is also possible for up to 20 wt. % of a permeation-promoting auxiliary to be added.

The formula constituents identified are defined in more detail as follows:

A:

Phase-separating block copolymers, especially A-B or A-B-A block copolymers or mixtures thereof based on poly(styrene-b-isoprene-b-styrene) (SIS), poly(styrene-b-isoprene-b-styrene) (SBS) or poly[styrene-b-(ethylene-random-butylene)-b-styrene] (SEBS). The total styrene fraction should always be lower than 35 wt. %. Preference is given to styrene fractions between 5% and 30%, since a lower styrene fraction makes the adhesive more conforming. Of particular advantage is the targeted blending of di-block and tri-block copolymers, preference being given to a di-block copolymer fraction of less than 80 wt. %.

B:

Tackifiers, examples being all known tackifying polymers, from the group, for example, of the polyisoprenes, polybutadienes and polyacrylates, and also tackifying resins. Here it is possible, without exception, to use all of the tackifier resins already known and described in the literature. Representatives that may be mentioned include the rosins, their disproportionated, hydrogenated, polymerized and esterified derivatives and salts, the aliphatic and aromatic hydrocarbon resins, terpene resins and terpene-phenolic resins. Any desired combinations of these and additional resins may be used in order to adjust the properties of the resulting adhesive in accordance with what is desired. Express reference may be made to the representation of the state of the art in the “Handbook of Pressure Sensitive Adhesive Technology” by Donatas Satas (van Nostrand, 1999).

C:

Plasticizers, all known plasticizing substances, and also pharmaceutical auxiliaries. They include, among others, the paraffinic and naphthenic oils (functionalized) oligomers such as oligobutadienes, oligoisoprenes, liquid nitrile rubbers, liquid terpene resins, vegetable and animal oils and fats, fatty acid esters, phthalates, alcohols and functionalized acrylates.

D:

Stabilizers, all known substances which prevent or retard the thermal or oxidative degradation of the water-insoluble, adhesive, active ingredient matrix. These may be, for example, representatives from the groups of the sterically hindered phenols, the thioorganic compounds, here preferably the derivatives of thiopropionic acid, the phosphorus compound classes of phosphites and phosphonites, or mixtures thereof.

E:

Active pharmaceutical ingredients are substances which in human or animal organisms serve to prevent, heal, alleviate or detect diseases. The active pharmaceutical ingredients employed may include both systemically active and locally active ingredients. Preference is given to using active ingredients with a topical action.

By way of example mention may be made here of salicylic acid (2-hydroxybenzoic acid).

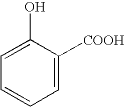

The production of the active ingredient matrix takes place preferably in a process in which all components of the adhesive matrix are homogenized in the melt, the addition of solvent being foregone.

The advantage of foregoing the use of solvent lies essentially in the simplification of the coating operation. The avoidance of flammable solvents does away with the need for the dryer unit, with its high energy consumption for the evaporation and recovery of the solvents, and with the need to use explosion-protected units. Hot melt coating units are compact and permit much higher coating speeds. The technology is an environment-friendly one in which there are no solvent emissions. Furthermore, no unwanted solvent residues remain in the self-adhesive composition. This is the reason for the reduction in the allergenic potential of the product.

For solvent-free compounding the prior art makes use predominantly of block copolymers having polystyrene block fractions, or natural and/or synthetic rubbers.

Owing to the high molecular mass fractions in natural rubber (with M_{w}≧3*10⁵ g/mol), solvent-free natural rubber self-adhesive compositions cannot be processed by the hot melt adhesive technology, or else the rubbers used must be reduced in their molecular weight (broken down) severely prior to processing.

The deliberate industrial process of rubber breakdown under the combined action of shearing stress, temperature and atmospheric oxygen is referred to in the technical literature as mastication and is generally carried out in the presence of chemical auxiliaries, which are known from the technical literature as masticating agents or peptizers, or, more rarely, as “chemical plasticizing agents”.

The mastication step is necessary in rubber technology in order to make it easier to integrate the additives.

According to Römpp (Römpp Lexikon Chemie—Version 2.0, Stuttgart/New York: Georg Thieme Verlag 1999) mastication is a term used in rubber technology for the breaking down of long-chain rubber molecules in order to increase the plasticity and/or reduce the (Mooney) viscosity of rubbers. Mastication is carried out by treating rubber, especially natural rubber, in kneading apparatus or between rolls at very low temperatures in the presence of mastication aids (masticating auxiliaries). The high mechanical forces which act here lead to a “tearing apart” of the rubber molecules, with the formation of macroradicals, whose recombination is prevented by reaction with atmospheric oxygen. Mastication aids such as aromatic or heterocyclic mercaptans and their zinc salts or disulfides accelerate the mastication process by promoting the formation of primary radicals. Activators such as metal (iron, copper, cobalt) salts of tetraazaporphyrins or phthalocyanines enable the mastication temperature to be lowered. For the mastication of natural rubber, mastication aids are used in amounts of about 0.1 to 0.5 wt. % in the form of masterbatches, which facilitate uniform distribution of this small amount of chemicals within the rubber composition.

Mastication must be clearly distinguished from the breakdown known as degradation which results in the course of all of the standard solvent-free polymer technologies, such as compounding, conveying and coating in the melt.

Degradation is a collective term for various processes which alter the appearance and properties of plastics. Degradation may be caused, for example, by chemical, thermal, oxidative, mechanical or biological influences or else by the effect of rays (such as (UV) light). Consequences are, for example, oxidation, chain scission, depolymerization, crosslinking and/or elimination of side groups of the polymers. The stability of polymers with respect to degradation may be increased by using additives, as for example by adding stabilizers such as antioxidants or light stabilizers.

Various routes have been described to the solvent-free preparation and processing of tacky rubber adhesives.

Canadian patent CA 698 518 describes a process for producing a composition by adding high fractions of plasticizer and/or by simultaneous strong mastication of the rubber. Although this method may be used to obtain adhesives having an extremely high tack, a user-compatible shear strength can be achieved only to a limited extent, even with a relatively high level of subsequent crosslinking, owing to the relatively high plasticizer fraction or else to the severe breakdown in molecular structure of the elastomer to a molecular weight average of Mw≦1 million.

The use of polymer blends, where, besides non-thermoplastic natural rubber, use is also made of block copolymers, in a ratio of approximately 1:1, is essentially an unsatisfactory compromise solution, since it results neither in high shear strengths when the self-adhesive tapes are used at relatively high temperatures nor in significant improvements relative to the properties described in the patent.

The use of exclusively non-thermoplastic rubbers as an elastomer component in the formulation of adhesives, with the existing cost advantage possessed, for example, by natural rubbers over the standard commercial block copolymers, and the outstanding properties, particularly the shear strength of natural rubber and of corresponding synthetic rubbers, is also set out in detail in WO 94 11 175 A1, WO 95 25 774 A1, WO 97 07 963 A1 and, correspondingly, U.S. Pat. No. 5,539,033 and U.S. Pat. No. 5,550,175.

In these cases, the additives customary in adhesive technology, such as tackifier resins, plasticizers and fillers, are described.

The production method disclosed in each case is based on a twin-screw extruder, which permits compounding to a homogeneous adhesive blend with the chosen operating regime, involving mastication of the rubber and subsequent gradual addition of the individual additives with an appropriate temperature regime.

The mastication step of the rubber, which precedes the actual production operation, is described at length. It is necessary and characteristic of the method chosen, since with the technology chosen therein it is indispensable for the subsequent integration of the further components and for the extrudability of the blended composition. Also described is the feeding in of atmospheric oxygen, as recommended by R. Brzoskowski, J. L. and B. Kalvani in Kunststoffe 80 (8), (1990), p. 922 ff., in order to accelerate mastication of the rubber.

This procedure makes it absolutely necessary to practice the subsequent step of curing by electron beam crosslinking (EBC), and to use reactive ingredients as EBC promoters in order to achieve an effective crosslinking yield.

Both method steps are described in the aforementioned documents, but the EBC promoters chosen also tend toward unwanted chemical crosslinking reactions at elevated temperatures; this limits the use of certain tackifying resins and the use of the self-adhesive compositions produced, particularly for pharmaceutical applications.

The thermoplastic properties of the adhesives of the invention permit their mastication-free processing. It has been found particularly advantageous for this purpose to use a twin-screw extruder having at least one metering port, preferably between two and seven, and at least one devolatilization port, as the continuously operating apparatus. The twin-screw extruder allows short mixing times and hence particularly gentle processing with the composition, especially of thermally sensitive components employed.

The self-adhesive composition of the invention can be prepared, for example, in accordance with different process variants. In the compounding step the adhesive of the invention is prepared solventlessly, preferably in a twin-screw extruder, from styrene block copolymers, one or more active pharmaceutical ingredients and the required adjuvants, such as low molecular mass SBC, fillers, plasticizers, tackifiers, resins and/or additives. The active pharmaceutical ingredients may be added immediately at the beginning of the process, or else—depending on the sensitivity of the active ingredient—can be added to the twin-screw extruder only at a later point in time. The addition may be made in neat form or in dissolved form.

Variant A

a) feeding an initial batch comprising SBC, optionally a release aid, and at least one pharmaceutical active ingredient into the feeding section of the apparatus,
   optionally, feeding low molecular weight SBC, fillers, plasticizers, tackifiers, resins and/or additives,
b) transferring the feed components of the self-adhesive composition from the feeding section to the compounding section,
c) optionally, adding the components of the self-adhesive composition that have not been introduced in the feeding section, such as low molecular weight SBC, fillers, plasticizers, tackifiers, resins and/or additives, to the compounding section,
d) optionally, adding further pharmaceutical active ingredients to the compounding section of the apparatus, and
e) preparing a homogeneous self-adhesive composition in the compounding section.

Additionally the self-adhesive compositions of the invention may be prepared, for example, in accordance with the following variant.

Variant B

a) feeding SBC, optionally with a release aid, into the feeding section of the apparatus, optionally, feeding low molecular weight SBC, fillers, plasticizers, tackifiers, resins and/or additives,
b) transferring the feed components of the self-adhesive composition from the feeding section to the compounding section,
c) adding at least one pharmaceutical active ingredient to the compounding section and, optionally, adding the components of the self-adhesive composition that have not been introduced in the feed section, such as low molecular weight SBC, fillers, plasticizers, tackifiers, resins and/or additives, to the compounding section, and
d) preparing a homogeneous self-adhesive composition in the compounding section.

The addition of at least one pharmaceutical active ingredient to the compounding section, and the possibly necessary addition to the compounding section of those components of the self-adhesive composition that have not been introduced in the feed section, such as low molecular weight SBC, fillers, plasticizers, tackifiers, resins and/or additives, may take place over the entire length of the compounding section. In particular a plurality of metering points are possible, so that each individual component may be metered in specifically via a separate feed opening depending on the requirements of the operating regime.

Moreover, within the apparatus a temperature of 85-120° C. should be maintained, preferably 85-110° C., more preferably 85-100° C., in order to rule out thermal damage to, in particular, the active ingredient or ingredients.

In one advantageous embodiment of the process there is a melt pump or an extruder for conveying the self-adhesive compositions arranged between the apparatus and the coating device.

In the second process step, which takes place advantageously in combination with the compounding step in the twin-screw extruder, the self-adhesive composition prepared in accordance with the invention, for the purpose of producing the active ingredient bandage systems of the invention, is coated solventlessly onto a backing in web form, onto a release film or onto a release paper, using an applicator unit. Coating may take place over the full area or else partially.

In order to obtain a defined, air-bubble-free application of composition onto the web material it is advantageous for the self-adhesive composition to be subjected to devolatilization before it enters the coating unit, something which is particularly important when inert gases are used during the compounding operation in the twin-screw extruder.

Additionally it is possible for devolatilization to take place under the influence of subatmospheric pressure or to the atmosphere, preferably in screw machines.

Various methods are suitable for coating onto web materials. Solvent-free self-adhesive compositions may be coated by means of an extrusion die downstream of the twin-screw extruder. For building up the pressure for die coating, single-screw extruders and/or melt pumps are particularly preferred, so that the web-form backing materials may be coated with application rates with a very low breadth of fluctuation.

A further possibility for coating web-form backing materials with the active ingredient self-adhesive composition prepared by inventive processes is the use of roll coating applicator units or multi-roll coating calenders, consisting preferably of at least two coating rolls, the self-adhesive composition being shaped to the desired thickness as it passes through one or more roll nips prior to transfer to the web-form material. This coating method is particularly preferred when coating with extrusion dies alone no longer provides the required accuracy in application rate.

Depending on the nature of the web-form backing material to be coated, coating may take place by a co-rotational or counter-rotational method.

Coating is possible on roll coating applicator units or multi-roll coating calenders at temperatures below 120° C., so that it is possible to coat self-adhesive compositions which include thermally sensitive active ingredients. For the purpose of increased gas bubble freedom in the coated adhesive, it is possible to install a vacuum devolatilizer, for example a vacuum chamber, a devolatilizing extruder, air knives or the like between the twin-screw extruder and the applicator unit.

As a result of this method there is no property-impairing mastication of the SBC, since the melting of the physically crosslinked styrene domains takes place shortly after the SBC is introduced.

The twin-screw extruder has one or, preferably, two or more separate temperature-control or cooling circuits in order to permit a temperature regime which allows the use of thermally sensitive pharmaceutical active ingredients. In cases where this is unnecessary the temperature-control circuits may also be combined with one another in order to minimize the number of temperature-control devices.

This process permits the preparation of self-adhesive compositions of the invention with pharmaceutical active ingredients and, in particular through the use of an extruder in integration with a downstream coating unit for the purpose of producing self-adhesive articles, which are used in turn to produce plasters or bandages, while achieving particular cost advantages.

The adhesives of the invention are prepared substantially under the process steps already described, which can optionally be carried out under an inert gas atmosphere in order to prevent oxidative polymer degradation.

The matrix is distinguished by outstanding properties of adhesion to the skin, by easy and painless redetachability, and, in particular, by its extremely low potential for inducing instances of skin irritation. The production operation takes place completely without solvents.

If desired, the open adhesive side, that intended for application to the skin, can be lined with a redetachable, covering protective layer.

### BRIEF DESCRIPTION OF THE DRAWINGS

In the drawings, the Figure shows a schematic representation of a system for carrying out an embodiment of the process of the present invention.

### DETAILED DESCRIPTION OF THE INVENTION

### EXAMPLE 1

A prototype of a self-adhesive composition doped with active ingredient was prepared solventlessly and continuously using a twin-screw extruder, employing the following exemplary formula (all figures in parts per hundred (phr) based on the sum of the block copolymer fraction):

| | | |
|---|---|---|
| A) | 100.0 | phr A-B/A-B-A block copolymer, consisting of hard and |
| | | soft segments, with a ratio of A-B-A to A-B of 29:21 |
| | | and a styrene content of 15% by mass (Vector 4114, Dexco) |
| C) | 33.0 | phr polyterpene resin (Sylvares TR 7115, Arizona Chemical) |
| D) | 41.9 | phr rosin ester resin (Foral 90, Eastman) |
| E) | 25.6 | phr hydrocarbon resin (Wingtack 95, Goodyear) |
| F) | 23.6 | phr mineral oil (Whitemor WOM 14, Castrol Ltd.) |
| H) | 8.9 | phr aging inhibitor (Irganox 1010, Ciba Specialty Chemicals) |
| I) | 259.6 | phr salicylic acid DAB [German pharmacopoeia] as active |
| | | ingredient. |

### EXAMPLE 2

A doped self-adhesive composition was prepared as in Example 1 in accordance with the following exemplary formula (all figures in parts per hundred (phr) based on the sum of the block copolymer fraction):

| | | |
|---|---|---|
| A) | 100.0 | phr A-B/A-B-A block copolymer, consisting of hard and |
| | | soft segments, with a ratio of A-B-A to A-B of 83:17 |
| | | and a styrene content of 15% by mass (Kraton D-1107, |
| | | Kraton) |
| C) | 67.7 | phr polyterpene resin (Sylvares TR 7115, Arizona Chemical) |
| D) | 86.6 | phr hydrocarbon resin (Wingtack 95, Goodyear) |
| F) | 57.1 | phr wool wax DAB [German pharmacopoeia] |
| H) | 11.5 | phr aging inhibitor (Lowinox 22M46, Great Lakes Chemical |
| | | Corp.) |
| I) | 226.4 | phr salicylic acid DAB [German pharmacopoeia] as active |
| | | ingredient. |

### EXAMPLE 3

A further prototype of a self-adhesive composition doped with active ingredient was prepared solventlessly and continuously with the same procedure as in Example 1. In this case the following exemplary formula (all figures in parts per hundred (phr) based on the sum of the block copolymer fraction) was used:

| | | |
|---|---|---|
| A) | 65.3 | phr A-B-A block copolymer, consisting of hard and soft |
| | | segments, having a styrene content of 15% by mass |
| | | (Vector 4114, Dexco) |
| B) | 34.7 | phr A-B/A-B-A block copolymer, consisting of hard and |
| | | soft segments, having a ratio of A-B-A to A-B of 41:9 |
| | | and a styrene content of 15% by mass (Vector 4113, |
| | | Dexco) |
| C) | 74.3 | phr rosin ester resin (Foral 90, Eastman) |
| F) | 28.1 | phr of beeswax DAB [German pharmacopoeia] |
| G) | 173.2 | phr wool wax DAB [German pharmacopoeia] |
| H) | 14.5 | phr aging inhibitor (Lowinox 22M46, Great Lakes Chemical |
| | | Corp.) |
| I) | 551.2 | phr salicylic acid DAB [German pharmacopoeia] as active |
| | | ingredient. |

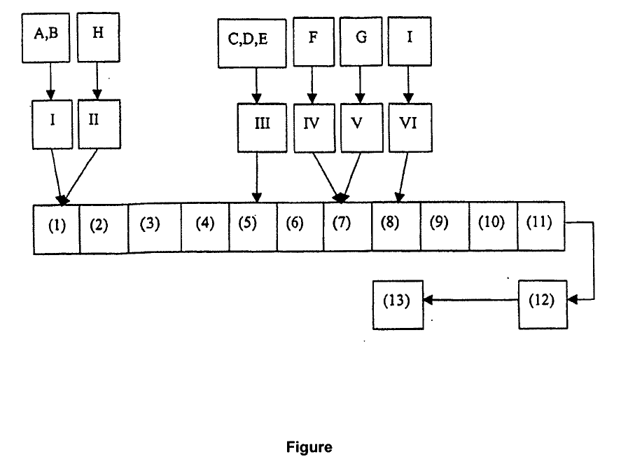

FIG. 1 shows a schematic overview of the system used to implement the process.

Raw materials A, B and H were each supplied via a gravimetric metering system (I) and (II) to the feed section of a twin-screw extruder.

The material was supplied via a first conveying process zone (1) to further zones (2)-(4), which mixed the material.

Components C/D/E were metered gravimetrically (III) into the conveying process zone (5), depending on formula. This was followed by mixing and conveying (6).

Thereafter came zone (7), which conveyed the material and to which components F/G were metered via volumetrically operating gear pumps (IV) and (V). Subsequently the material was mixed again.

This was followed by zone (8), which conveyed the material and to which component I was metered via a gravimetric metering system (VI).

In zones (9)-(11) the material was mixed and conveyed.

Subsequently the self-adhesive composition was shaped by way of a 350 mm slot die (12) and extruded. Calendering took place in a calender unit (13), along with lamination to two PET films.

The rotary speed of the extruder was between 100 and 150 rpm. At the exit from the extruder the composition had a temperature of between 90° C. and 100° C.

1. An active ingredient containing bandage for the controlled delivery of at least one pharmaceutically active ingredient to the skin, wherein the bandage comprises a flexible cover layer and a water-insoluble, adhesive styrene block copolymer (SBC)-based matrix that comprises at least 34% by weight of at least one pharmaceutically active ingredient, based on a total weight of a matrix composition.

2. The bandage of claim 1, wherein the SBC comprises at least one of an A-B block copolymer and an A-B-A block copolymer and A is a hard phase which comprises predominantly polystyrene and derivatives thereof and B is a soft phase which comprises predominantly units of at least one of ethylene, propylene, butylene, butadiene and isoprene.

3. The bandage of claim 2, wherein the at least one of an A-B block copolymer and an A-B-A block copolymer comprises less than 35% by weight of styrene units.

4. The bandage of claim 2, wherein the at least one of an A-B block copolymer and an A-B-A block copolymer comprises from 5% to 30% by weight of styrene units.

5. The bandage of claim 2, wherein less than 80% by weight of the at least one of an A-B block copolymer and an A-B-A block copolymer consists of an A-B block copolymer.

6. The bandage of claim 1, wherein the matrix comprises from 4% to 55% by weight of SBC.

7. The bandage of claim 2, wherein the matrix comprises from 5% to 50% by weight of SBC.

8. The bandage of claim 3, wherein the matrix comprises from 7% to 45% by weight of SBC.

9. The bandage of claim 1, wherein the matrix comprises up to 70% by weight of the at least one pharmaceutically active ingredient.

10. The bandage of claim 6, wherein the matrix comprises from 36% to 65% by weight of the at least one pharmaceutically active ingredient.

11. The bandage of claim 8, wherein the matrix comprises from 37% to 62% by weight of the at least one pharmaceutically active ingredient.

12. The bandage of claim 1, wherein the bandage comprises from 4% to 55% by weight of SBC, from 5% to 55% by weight of tackifiers, less than 51% by weight of plasticizers, less than 5% by weight of stabilizers, and up to 70% by weight of the at least one pharmaceutically active ingredient.

13. The bandage of claim 1, wherein the at least one pharmaceutically active ingredient comprises an active ingredient that is locally active.

14. The bandage of claim 1, wherein the active ingredient comprises salicylic acid.

15. The bandage of claim 1, wherein all components of the matrix have been melt-homogenized without use of solvents.

16. A process for a solvent-free and mastication-free production of a self-adhesive styrene block copolymer (SBC)-based composition which comprises at least one pharmaceutically active ingredient in an amount of at least 34% by weight based on a total weight of SBC-based composition in a continuously operating apparatus which comprises a feeding section and a compounding section, the process comprising
(a) feeding an initial batch which comprises SBC, at least a part of the at least one pharmaceutically active ingredient and, optionally, at least a part of any further components of the composition into the feeding section of the apparatus;
(b) transferring the initial batch from the feeding section to the compounding section of the apparatus;
(c) optionally, adding any remaining part of the at least one pharmaceutically active ingredient and any remaining part of the further components of the composition which have not been added to the feeding section to the compounding section; and
(d) producing a homogeneous self-adhesive composition in the compounding section.

17. The process of claim 16, wherein the further components of the composition are selected from one or more of low molecular weight SBCs, fillers, plasiticizers, tackifiers, resins, release aids and additives.

18. The process of claim 16, wherein the apparatus comprises a twin-screw extruder.

19. The process of claim 18, wherein the twin-screw extruder comprises at least one metering port and at least one devolatilization port.

20. The process of claim 18, wherein the twin-screw extruder comprises from two to seven metering ports.

21. The process of claim 18, wherein a temperature inside the apparatus is from 85° C. to 120° C.

22. The process of claim 21, wherein the temperature is up to 110° C.

23. The process of claim 21, wherein the temperature is up to 100° C.

24. A process for a solvent-free and mastication-free production of a self-adhesive styrene block copolymer (SBC)-based composition which comprises at least one pharmaceutically active ingredient in an amount of at least 34% by weight based on a total weight of SBC-based composition in a continuously operating apparatus which comprises a feeding section and a compounding section, the process comprising
(a) feeding an initial batch which comprises SBC and, optionally, at least a part of any further components of the composition with the exception of the at least one pharmaceutically active ingredient into the feeding section of the apparatus;
(b) transferring the initial batch from the feeding section to the compounding section of the apparatus;
(c) adding the at least one pharmaceutically active ingredient and, optionally, any remaining part of the further components of the composition which has not been added to the feeding section to the compounding section;
(d) producing a homogeneous self-adhesive composition in the compounding section.

25. The process of claim 24, wherein the further components of the composition are selected from one or more of low molecular weight SBCs, fillers, plasiticizers, tackifiers, resins, release aids and additives.

26. The process of claim 24, wherein the apparatus comprises a twin-screw extruder.

27. The process of claim 26, wherein the twin-screw extruder comprises at least one metering port and at least one devolatilization port.

28. The process of claim 27, wherein the twin-screw extruder comprises from two to seven metering ports.

29. The process of claim 24, wherein a temperature inside the apparatus is from 85° C. to 120° C.

30. The process of claim 29, wherein the temperature is up to 110° C.

31. The process of claim 30, wherein the temperature is up to 100° C.

32. A process for producing a self-adhesive, active ingredient containing matrix bandage, which process comprises carrying out the process of claim 16, transferring the self-adhesive composition to a coating device and applying the self-adhesive composition to a carrier material selected from a web material, a release film and a release paper.

33. The process of claim 32, wherein the coating device comprises an extrusion die.

34. The process of claim 32, wherein the coating device comprises one of a roll unit and a calender unit.

35. The process of claim 32, wherein a thickness of the self-adhesive composition on the carrier material is adjusted to from 10 μm to 2,000 μm.

36. The process of claim 35, wherein the thickness is from 100 μm to 500 μm.

37. A process for producing a self-adhesive, active ingredient containing matrix bandage, which process comprises carrying out the process of claim 24, transferring the self-adhesive composition to a coating device and applying the self-adhesive composition to a carrier material selected from a web material, a release film and a release paper.

38. The process of claim 37, wherein the coating device comprises an extrusion die.

39. The process of claim 37, wherein the coating device comprises one of a roll unit and a calender unit.

40. The process of claim 37, wherein a thickness of the self-adhesive composition on the carrier material is adjusted to from 10 μm to 2,000 μm.

41. The process of claim 40, wherein the thickness is from 100 μm to 500 μm.

42. An active ingredient containing self-adhesive SBC-based composition which is obtainable by the process of claim 16.

43. An active ingredient containing self-adhesive SBC-based composition which is obtainable by the process of claim 24.

44. A self-adhesive, active ingredient containing matrix bandage which is obtainable by the process of claim 32.

45. A self-adhesive, active ingredient containing matrix bandage which is obtainable by the process of claim 37.
